(19) United States
(12) Patent Application Publication
(10) Pub. No.: US 2026/0184865 A1
(43) Pub. Date: Jul. 2, 2026

(54) METHOD FOR PREPARING AN ANTHROPOGENIC TARGET SUBSTANCE

(71) Applicant: IST Innuscreen GmbH, Berlin (DE)

(72) Inventors: Timo Hillebrand, Hönow (DE); Elmara Graser, Berlin (DE); Wiebke Jacobi, Falkensee (DE)

(21) Appl. No.: 19/117,908

(22) PCT Filed: Sep. 13, 2023
(86) PCT No.: PCT/EP2023/075134

§ 371 Date: Apr. 2, 2025

(30) Foreign Application Priority Data
Oct. 6, 2022 (DE) 10 2022 125 807.6

(51) Int. Cl. C08J 5/02; B82Y 5/00; B82Y 40/00; C08K 5/29

(52) U.S. Cl. CPC C08J 5/02; B82Y 5/00; B82Y 40/00; C08K 5/29; C08J 2307/02

(57) ABSTRACT

A method for preparing an anthropogenic target substance includes: producing an emulsion or suspension of the target substance, the emulsion or suspension containing particles and/or particulates of the target substance with an average particle size or particulate size in the nanometer range; concentrating the emulsion or suspension by: a) adding a superabsorber to an initial first liquid volume of the emulsion or suspension or adding the first liquid volume to the superabsorber, and b) incubating the mixture formed from the superabsorber and the first liquid volume for a first period of time; and removing a second liquid volume from the liquid portion of the mixture present after incubation.

The invention relates to a method for preparing an anthropogenic target substance.

Nanoparticles and nanoparticulates are playing an increasingly important role in our lives. This concerns, for example, the use, and in this connection also the production, of these particles and particulates for a variety of applications in the pharmaceutical industry (for example, in connection with encapsulated mRNA active ingredients), food technology, or the electrical engineering or electronics industry (for example, in connection with quantum dots).

Due to their small size and the associated changes in physical characteristics, nanoparticles or nanoparticulates can be used to transport drugs to desired organs, since they can penetrate the blood-brain barrier (e.g., suitable for use in pharmacology) and the skin (e.g., suitable for use in the cosmetics industry). Nanoparticles or nanoparticulates are also used for diagnostic uses—for example, for color detection in rapid tests based upon colloidal gold. They also stabilize food and thereby ensure longer shelf lives.

Nanoparticles or nanoparticulates are almost exclusively anthropogenic substances. This means that the nanoparticles or nanoparticulates have been produced by humans through industrial, commercial, and communal processes and not, for example, by biological organisms. Possible components include natural polymers such as albumin, but also biocompatible, synthetic polymers such as polymethyl methacrylate, polyalkyl cyanoacrylate, and copolymers, as well as various inorganic or organic particles.

Most of the manufacturing methods for nanoparticles are based upon sol gel methods, emulsion polymerization, interfacial polymerization, and similar methods. At the end of the process, a nanoparticle emulsion is present in a suitable solvent.

The concentration of the desired nanoparticles in the solvent naturally fluctuates during the production process and is often too low. The produced nanoparticles or nanoparticulates must therefore be concentrated. However, due to the small size, this poses a problem. Concentration is usually performed by ultracentrifugation. This method is complex and can often be performed with—for large-scale industry—only a relatively small volume. Some nanoparticles or nanoparticulates might also be destroyed during centrifugation. Filtration is not an option because of the minute size of the nanoparticles or nanoparticulates.

The object of the invention is to present a production method for an anthropogenic target substance containing nanoparticles or nanoparticulates, which includes a simple, rapid, and universally applicable concentration method of the nanoparticles or nanoparticulates.

This object is achieved in a surprisingly simple and universally applicable manner by means of the method defined in claim 1. Advantageous embodiments are listed in the dependent claims.

The solution is based upon the use of so-called superabsorbers, with which any aqueous liquid, e.g., an emulsion or suspension, can be processed in order to concentrate the nanoparticles or nanoparticulates contained in the liquid.

Superabsorbers (superabsorbent polymers, SAP) are plastics which are capable of absorbing a multiple of their own weight in polar liquids. These are mainly water or aqueous solutions. When the liquid is absorbed, the superabsorber swells and forms a hydrogel. Hydrogels can be formed by all cross-linked polymers which are polar (e.g., polyacrylamide, polyvinylpyrrolidone, amylopectin, gelatin, cellulose). Usually, however, a copolymer of acrylic acid (propene acid, H2C═CH—COOH) or sodium acrylate (sodium salt of acrylic acid, H2C═CH—COONa) on the one hand and acrylamide on the other is used, wherein the ratio of the two monomers to one another can vary. In addition, a so-called core cross-linker (CXL) is added to the monomer solution and connects the formed long-chain polymer molecules with each other in places by chemical bridges, also known as cross-linking. The polymer is water-insoluble because of these bridges. This so-called base polymer is possibly subjected to a so-called surface cross-linking (SXL). A further chemical is applied here to the surface of the particles which, by heating, links a second network only to the outer layer of the grain. This shell supports the swollen gel in order to also hold together under external loading (movement, pressure).

The product is traditionally used, for example, as white granules having particulate sizes of 100-1,000 μm. It is mainly used in baby diapers, sanitary napkins, incontinence care, bandages, and, in small amounts, also in cable sheathings for deep-sea cables. Further areas of application are so-called gel beds, gel-forming extinguishing agents in firefighting, as a mechanical stabilizer for cut flowers in a vase, or as additive for plant soil in order to save upon water on an ongoing basis. However, due to its better environmental compatibility, potassium-neutralized acrylic acid is used here. In the form of spherical particles, the use of superabsorbers as a toy is known, with designations such as “Wasserperlen,” “Aqua Beads,” or “Water Beads.” These are superabsorbers which are commercially available in the form of spheres of variable sizes (submillimeters to centimeters).

The invention was based upon the following unexpected observation: A water sample was mixed with fluorescent nanoparticulates with an average particle or particulate size of 30 nm. After adding commercially available water pearl beads and an incubation period during which the beads swelled to several times their original volume, it was shown that the nanoparticulates were not absorbed by the superabsorbers, but were concentrated in the remaining residual liquid.

This observation shows that, by using superabsorbers, and in particular, superabsorbers that are commercially available in the form of so-called water pearl beads, a wide variety of nanoparticulates can be easily and quickly concentrated in a liquid sample. In industrial manufacturing processes of nanoparticulates or nanoparticles, these can be made available in higher concentrations through this simple possibility of concentration (e.g., no complex and inefficient ultracentrifugation or ultrafiltration is necessary).

On the basis of this observation, it was possible to achieve the object of the invention.

The method according to the invention for producing an anthropogenic target substance comprises:
producing an emulsion or suspension of the target substance, wherein the emulsion or suspension contains particles and/or particulates of the target substance with an average particle size or particulate size in the nanometer range, and
concentrating the emulsion or suspension by:
   a) adding a superabsorber to an initial first liquid volume of the emulsion or suspension or adding the first liquid volume to the superabsorber,
   b) incubating the mixture obtained by mixing the superabsorber and the liquid volume over a first period of time, and
   c) removing a second liquid volume from the liquid portion, present after incubation, of the mixture.

“Anthropogenic substances” are substances that are not created by nature, but by humans—for example, through industrial, commercial, or communal processes. They include, for example, plastics, but also pesticides, pharmaceuticals, personal care products, and industrial chemicals, as well as their degradation products and metabolites. Biomolecules that are produced, for example, by microorganisms (e.g., enzymes, DNA/RNA fragments, etc.) and have similar sizes in the nanometer range do not fall under the particulates or particles of anthropogenic substances mentioned in this application.

In the method according to the invention indicated above, the second liquid volume, which is removed from the liquid portion, present after incubation, of the mixture of liquid and superabsorber, can be the entire remaining liquid portion. However, it is also possible to remove only a partial volume of the given liquid portion as the second liquid volume.

In the second liquid volume, the nanoparticles or nanoparticulates are present in a high concentration. The concentration or the presence of the correct substance can be examined in a subsequent analysis, e.g., by means of a spectroscopic examination. However, the second liquid volume can also be further concentrated in a cascading method in one or more additional stages, e.g., by readdition of a superabsorber and/or readdition to a superabsorber and reincubation, or by means of a conventional method for concentrating target substances—for example, by one of the methods specified at the outset. If only a part of the liquid portion is removed as the second liquid volume, the target substance in the liquid portion of the mixture remaining after the removal can be further concentrated by reincubation over a second period of time. Both variants of the method can be performed repeatedly, so that a higher concentration of the target substance is obtained at each stage of the cascading concentration.

In an advantageous embodiment of the method, it is provided that the emulsion or suspension be produced by a sol gel method, an emulsion polymerization method, or an interfacial polymerization method. Alternatively, the emulsion or suspension is produced by a crystallization or a complex formation. These are established methods for producing nanoparticles or nanoparticulates, wherein they are present in an emulsion or suspension after completion of the processes.

It is advantageous, in a first stage, to reduce the initial sample volume by means of the method according to the invention using a superabsorber, and in a subsequent second stage to perform further concentration of the target substance by means of a conventional concentration technique—for example, filtration, ultrafiltration, precipitation reaction, ultracentrifugation, or coating by means of the method described in EP 2283026 B1. These known techniques can be used in already reduced liquid volumes significantly more efficiently than in more strongly diluted solutions. The method according to the invention is thereby suitable for significantly simplifying known methods for concentrating nanoparticles or nanoparticulates for large-volume sample liquids and/or sample liquids containing low concentrations of the target substance. As mentioned, in an advantageous embodiment, after removal of the first sample, the method can comprise a further concentration of the target substance in the removed first sample.

The further concentration of the target substance in the removed second liquid volume can be performed by means of a filtration, ultrafiltration, or precipitation reaction technique.

Alternatively, however, the further concentration of the target substance in the removed second liquid volume can also be performed again, and optionally in a cascading manner, once or multiple times by performing the following method steps:
adding a superabsorber to the first sample or adding the first sample to the superabsorber,
incubating the mixture, obtained by mixing the superabsorber and the second liquid volume, over a second period of time, and
removing a concentrated third liquid volume of the liquid portion, present after incubation, of the mixture.

As described above, in the first stage of the cascade, the concentrated third liquid volume that is removed from the liquid portion remaining after incubation can comprise the entire volume of the liquid portion. Alternatively, the concentrated third liquid volume can be a partial volume of the remaining liquid portion.

The target substance can be further concentrated in the concentrated third liquid volume or in a further concentrated third liquid volume obtained by further concentration, in particular, by means of a superabsorber, by means of a filtration, ultrafiltration, or precipitation reaction. This is advantageous if the volume of the concentrated first sample corresponds to only a few milliliters, e.g., 1 to 10 mL.

In the method according to the invention, the initially used first liquid volume, i.e., the emulsion or suspension, can contain a polar liquid, in particular, as a main constituent. In an advantageous embodiment of the method according to the invention, the liquid volume can contain a polar solvent, in particular, as a main constituent. For example, the liquid volume can consist of a polar solvent, at a mass fraction of at least 50%. The polar liquid or the polar solvent can be water, for example.

The target substance is a nanoparticle or nanoparticulate. Such a substance consists of an anthropogenic substance, in particular, of at least one natural polymer, of at least one biocompatible synthetic polymer, of an inorganic material, or of an organic material.

As mentioned, in an advantageous embodiment, the superabsorber can be a plastic or comprise a plastic which absorbs a proportion of the first liquid volume, e.g., a polar solvent contained in the first liquid volume, such as water, to form a gel or hydrogel. Advantageously, the plastic is selected such that it does not substantially absorb nanoparticulates or nanoparticles. This is the case, for example, in the above-mentioned superabsorbers made of the mentioned polymer and/or copolymer materials—for example, the commercially available Wasserperlen, Water Beads, etc.

The superabsorber can be used in the form of particles, e.g., as a powder, as granules, or in the form of geometric bodies, in particular, beads (spherical particles). It can thus be added to the liquid volume and/or the first sample in the form of such particles, or the liquid volume and/or the first sample can be added to the superabsorber in this form. The particles or beads can have a diameter between 100 to 5,000 μm.

Advantageously, the superabsorber is commercially available superabsorber spheres—for example, superabsorber beads available under the names “aqua beads”, “water beads”, “water pearls”, “aqua pearls”, “hydro beads”, and “gel beads”.

In an advantageous method embodiment, the volume of the liquid portion remaining after the step of incubating, and thereby the concentration of the target substance in the remaining liquid portion, is controlled by the length of the period or the periods of time of incubation, by the size and number of the superabsorber particles or superabsorber spheres added to the initially used first liquid volume of the sample liquid and/or of the first sample, and/or by the temperature prevailing during the incubation.

Subsequent physical detection methods for the qualitative and/or quantitative detection of the nanoparticles or nanoparticulates in the removed liquid volumes are also possible, for example, to determine whether certain nanoparticles or nanoparticulates are present in the corresponding concentrated liquid volume or how high their concentration is. In the emulsion or suspension, these would not be detectable using the physical detection method because their concentration is too low. A microscope is used for qualitative and/or quantitative determination. In this case, for example, the nanoparticles or nanoparticulates are counted in a section of defined size. Alternatively, a fluorescence measurement method or a spectroscopic measurement method is used. For this purpose, the nanoparticles or nanoparticulates must contain a fluorescent dye, which is added in particular, before concentration.

The invention is explained in more detail below with reference to the figures and some exemplary embodiments. These examples do not represent any limitation of the means and methods according to the invention. In the figures:

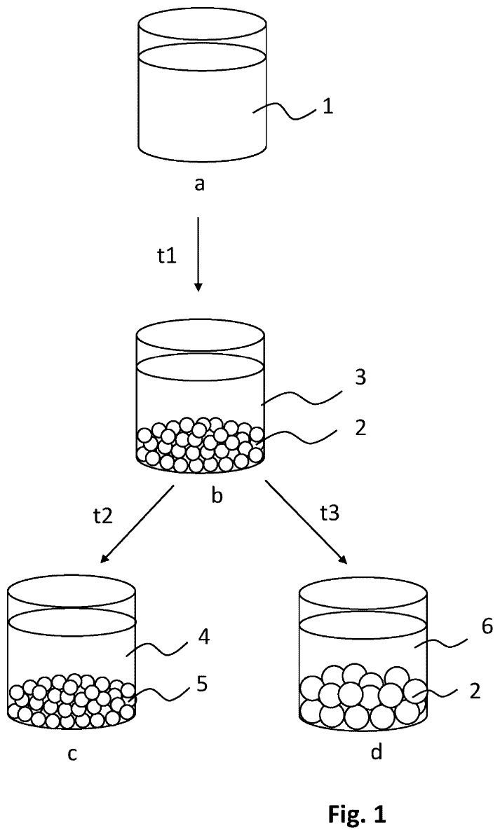

FIG. 1 is a schematic representation of the cascading concentration of a target substance in a liquid;
a) liquid before the addition of a superabsorber;
b) liquid after the addition of a superabsorber and incubating the mixture;
c) a second liquid volume removed from the mixture after adding a further superabsorber and incubating the mixture;
d) remaining mixture of liquid and superabsorber, optionally after removal of the second liquid volume and after reincubation;

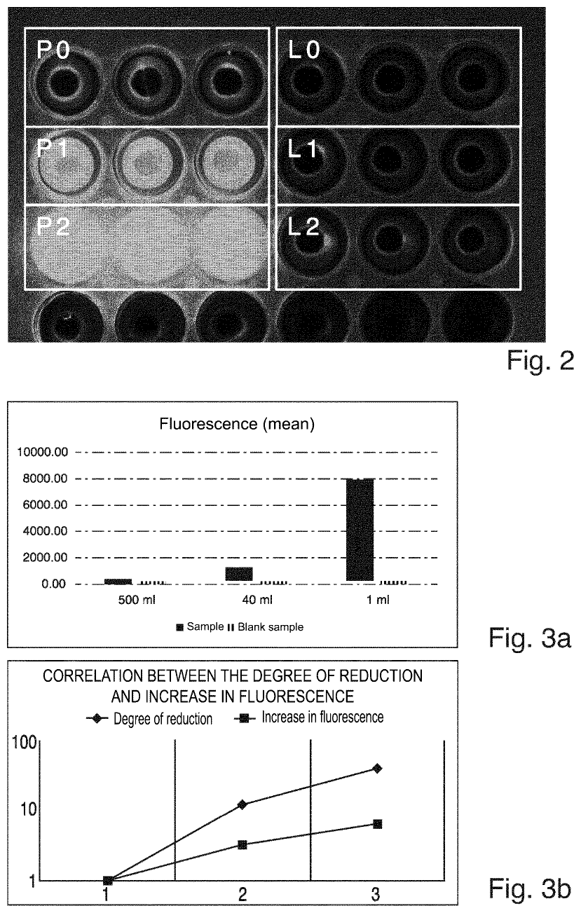

FIG. 2 is a representation of samples and blank samples, some of which were obtained by means of the concentration according to the invention and exposed to UV light; and

FIG. 3 shows evaluations of the measurement data:
(a) a graphical representation of the mean values of the measured values in a bar chart;
b) a correlation between the degree of concentration and the increase in fluorescence of the samples.

The use of superabsorbers for concentrating a target substance, in particular, nanoparticles or nanoparticulates, in a polar liquid as solvent, e.g., water, for concentrating the target substance in the liquid is very simple and universally usable. A suitable method is described briefly with reference to FIGS. 1a and b as follows:
1. addition of a superabsorber 2 to a volume of a liquid 1, in particular, an aqueous liquid, or alternatively: addition of the liquid 1 to a superabsorber 2;
2. incubation of the mixture of the liquid 1 and the superabsorber 2 over a first period of time t1 for reducing (reduction in volume) the liquid portion 3 of the mixture; and then
3. transferring at least one second liquid volume 4 of the liquid portion 3 of the mixture into a new vessel as a sample for further processing.

Further processing can, for example, be a quantitative and/or qualitative detection of the target substance in the liquid. The detection is performed, for example, using a spectroscopic or microscopic method.

The degree of concentration and the speed of this process can be controlled very precisely by means of the type of superabsorber used, by means of the amount thereof used, or by means of the incubation time and/or the incubation temperature.

With this method, the problem of concentration during the production of nanoparticles or nanoparticulates can be solved easily. The method shown in FIGS. 1a and b works without devices such as ultra-centrifuges, expensive ultrafiltration membranes, complex processes such as PEG precipitation, or, in general, precipitation reactions for concentrating nucleic acids, etc. In addition, the method can be used universally in relation to the type of nanoparticles or nanoparticulates. A further advantage is that the superabsorbers are non-toxic and safe and often also biodegradable. The method according to the invention makes it possible to greatly simplify the investigation of low-concentrated nanoparticulates or nanoparticles.

In the case of large-volume and/or strongly diluted liquids which contain the nanoparticulates or nanoparticles at a very low concentration, a cascading concentration of the target substance is possible. For example, in a first stage, the described method can be used with the aforementioned steps 1-3 in order to concentrate the target substance. In a second stage, the volume of the second liquid volume 4 can be reduced for reconcentration of the target substance. This can be done either by means of a conventional filtration or precipitation method, or other conventional methods. Alternatively, however, reconcentration of the target substance in the second liquid volume 4 can also take place, as shown in FIG. 1c, by adding fresh superabsorber 5 to the second liquid volume 4, or transferring the first sample 4 to a new superabsorber 5, and reincubating over a second period of time t2. Additionally or alternatively, the liquid portion 6 remaining in a mixture with the superabsorber 2 after removal of the second liquid volume 4 can be further reduced by incubating the mixture over a third period of time t3, as shown in FIG. 1d. This leads to further swelling with an increase in volume of the spheres consisting of the superabsorber 2, and to a further volume reduction in the liquid portion 6, which is accompanied by an increase in concentration of the target substance in the liquid portion 6.

In both alternative method paths, further cascading concentration stages can follow.

An exemplary embodiment of the invention will be described in detail below.

### EXEMPLARY EMBODIMENT: CONCENTRATION OF RHODAMINE B-FILLED LATEX NANOPARTICLES (DIA. 25.8 NM) IN A WATER SAMPLE

The latex nanoparticles were provided by the Fraunhofer Institute for Applied Polymer Research. The concentration of latex particles in the stock solution was 2.02 M %. The particles were added to a 500 ml water sample, and a 1:10,000 dilution was produced.

Subsequently, a superabsorber in the form of commercially available “Water Beads” was added to the water sample. During the concentration by the absorption of water into the water beads, samples P0, P1, P2, which each corresponded to different concentrations, were taken at different times.

The samples are divided into blank samples L0, L1, L2 for control (without latex nanoparticles) and samples P0, P1, P2 that contain the latex nanoparticles. Specifically, the samples are as follows:
Blank sample L0: ultrapure water without latex particles (500 mL)
Blank sample L1: concentration of 500 mL ultrapure water to 40 mL ultrapure water
Blank sample L2: further concentration of L1 to 1 mL ultrapure water
Sample P0: 1:10,000 dilution of the latex particle stock solution in 500 mL ultrapure water
Sample P1: concentration of 500 mL ultrapure water to 40 mL ultrapure water
Sample P2: further concentration of P1 to 1 mL ultrapure water

The particles in the respective samples were detected by measuring the fluorescence of the dye contained in the latex nanoparticles.

FIG. 1 shows a qualitative detection of the latex nanoparticles. For this purpose, the respective samples P0, P1, P2 and the blank samples L0, L1, L2 were each transferred in triplicate to a UV-transparent measuring plate. The measuring plate with the samples was then exposed to UV light. Exposure to UV light causes fluorescence excitation in those samples in which there are latex particles. No fluorescence is seen in the blank samples L0, L1, L2, and an increasing fluorescence is seen with increasing concentration of the samples P0, P1, P2, which contain the latex nanoparticles. Table 1 shows a quantitative measurement of the respective samples using a fluorescence measuring device. The measurements are performed by exciting the samples P0, P1, P2 and the blank samples L0, L1, L2 (each in triplicate) at an emission of 559 nm. Whereas the values of the blank samples L0, L1, L2 with the concentration remain consistently low, the values of the samples P0, P1, P2 that contain the nanoparticles increase proportionally to the degree of concentration.

TABLE 1

| | Fluorescence | | Fluorescence |
|---|---|---|---|
| Sample | measured values | Sample | measured values |
| P0 | 432.92/344.75/414.06 | L0 | 193.38/168.69/269.60 |
| P1 | 1,282.19/1,214.20/ | L1 | 220.20/187.81/257.12 |
| | 1,292.55 | | |
| P2 | 7,694.19/7,995.08/ | L2 | 237.47/257.65/234.28 |
| | 8,099.97 | | |

In FIG. 2, the measured values listed in Table 1 are graphically displayed or evaluated. In FIG. 2a), the mean values of the respective measured values of a sample are shown as a bar chart. In FIG. 2b), a correlation between the degree of concentration and the increase in fluorescence of samples P0 (“1”), P1 (“2”), and P2 (“3”) containing nanoparticles is shown.

The data from the experiment clearly show that the fluorescent latex nanoparticles of the sample do not diffuse into the utilized superabsorber (“Water Beads”) but remain in the external solution, and the concentration of the latex nanoparticles thereby increases continuously and in correspondence to the degree of concentration. This allows nanoparticles or nanoparticulates that are manufactured in an industrial process to be optimally concentrated. The industrial process includes, for example, a sol gel method, an emulsion polymerization method, or an interfacial polymerization method. This creates an emulsion or suspension consisting of a liquid portion and the nanoparticulates or nanoparticles. The nanoparticulates or nanoparticles have an average size of less than one micrometer. Alternatively, the suspension can also be produced by a crystallization or a complex formation. Adding the emulsion or suspension to the superabsorber results in the superabsorber absorbing the liquid of the emulsion or suspension, such that the nanoparticulates or nanoparticles are present in a high concentration in the remaining residue.

List of Reference Signs

| | |
|---|---|
| 1 | Initial first liquid volume |
| 2, 5 | Superabsorber |
| 3, 6 | Liquid portion of the mixture |
| 4 | Second liquid volume |
| t1, t2, t3 | Periods of time |
| L0, L1, L2 | Blank samples |
| P0, P1, P2 | Samples |

1-15. (canceled)

16. A method for producing an anthropogenic target substance, the method comprising:
producing an emulsion or suspension of the target substance, the emulsion or suspension containing particles and/or particulates of the target substance with an average particle or particulate size in a range of nanometers;
concentrating the emulsion or suspension by:
adding a superabsorber to an initial first liquid volume of the emulsion or suspension, or adding the first liquid volume to the superabsorber, to form a first mixture; and
incubating the first mixture for a first period; and
removing a second liquid volume from a liquid portion, which remains after incubation, of the first mixture.

17. The method according to claim 16, wherein the emulsion or suspension is produced by a sol gel method, an emulsion polymerization method, or an interfacial polymerization method.

18. The method according to claim 16, wherein the emulsion or suspension is produced by a crystallization or a complex formation.

19. The method according to claim 16, further comprising further concentrating the target substance in the removed second liquid volume.

20. The method according to claim 19, wherein the further concentration of the target substance in the removed second liquid volume is performed by a filtration, ultrafiltration, or precipitation reaction technique.

21. The method according to claim 19, wherein the further concentration of the target substance in the removed second liquid volume is performed, by:
adding a second superabsorber to the second liquid volume or adding the second liquid volume to the second superabsorber, to form a second mixture;
incubating the second mixture over a second period, wherein after incubation a concentrated liquid portion and the second superabsorber remain; and
removing a concentrated third liquid volume from the concentrated liquid portion, which remains after incubation, of the second mixture.

22. The method according to claim 21, wherein the target substance is further concentrated in the removed third liquid volume using a superabsorber.

23. The method according to claim 16, wherein the first liquid volume contains a polar liquid.

24. The method according to claim 23, wherein the polar liquid is water.

25. The method according to claim 23, wherein the particles or particulates of the target substance consist of at least one natural polymer, of at least one biocompatible synthetic polymer, of an inorganic material, or of an organic material.

26. The method according to claim 16, wherein the first superabsorber comprises a plastic material, which absorbs a portion of the first liquid volume such that a hydrogel is formed.

27. The method according to claim 26, wherein the plastic material does not absorb essentially any particles and/or particulates contained in the target substance.

28. The method according to claim 16, wherein the first superabsorber is in the form of particles, including powder, granules, spheres, or other geometric bodies.

29. The method according to claim 16, wherein the first superabsorber is in a form of commercially available “aqua beads”, “water beads”, “water pearls”, “aqua pearls”, “hydro beads”, or “gel beads”.

30. The method according to claim 21, wherein a volume of the liquid portion and/or the concentrated liquid portion remaining after incubation is controlled by at least one of: the length of the first period and/or the second period, respectively;
a type and/or amount of the first superabsorber and/or the second superabsorber, respectively; and
a temperature of the first and/or second mixture, respectively, present during incubation.

31. The method according to claim 30, wherein the first superabsorber and/or the second superabsorber are in the form of particles, including powder, granules, spheres, or other geometric bodies, and
wherein the volume of the liquid portion and/or the concentrated liquid portion remaining after incubation is controlled by the size and/or number of the particles.
